(19) United States
(12) Patent Application Publication
(10) Pub. No.: US 2006/0122470 A1
(43) Pub. Date: Jun. 8, 2006

(54) Systems and methods for data visualization

(75) Inventor: Thomas Schulz, Portola Valley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Sageera Institute LLC

(21) Appl. No.: 11/006,969

(22) Filed: Dec. 7, 2004

(51) Int. Cl. A61B 5/00

(52) U.S. Cl. 600/300

(57) ABSTRACT

Systems and methods consistent with embodiments of the present invention provide for a method for display of anthropometric measures on a plurality of scales, in an analog format, using a single pointer. In some embodiments, the plurality of data selected for display may comprise both raw and derived anthropometric data. In some embodiments, the raw data displayed may comprise the weight of a subject and the derived data displayed may comprise the BMI and the weight category of the subject. In some embodiments consistent with the present invention, the pointer may be aligned to a fixed scale corresponding to an anthropometric measure and other scales may be aligned with the pointer to correspond to the anthropometric measures represented by the individual scales. In some embodiments, some or all of the scales may be color-coded.

### BACKGROUND

1. Field of the Invention

The present invention relates to the field of data visualization and in particular, to systems and methods for the display of anthropometric data.

2. Description of Related Art

Anthropometric data, including both raw and derived data, is increasingly used by health care professionals and patients to help monitor, track and achieve health and wellness goals. Raw anthropometric data pertaining to a subject can be directly measured using a measuring device. Derived anthropometric measurements can be obtained by combining raw anthropometric measurements with each other, or with other data pertaining to a subject such as gender, race, or ethnicity.

For example, a derived anthropometric measure termed the Body Mass Index (“BMI”) is often used to calculate a subject's weight category. Knowledge of a subject's weight category is important in the diagnosis of obesity and associated health risks, such as diabetes and coronary heart disease. To calculate BMI using current standardized formulas, a patient's height and weight are measured, and BMI is then calculated as:

$$BMI = \frac{Weight}{{Height}^{2}}$$

Thus, health care professionals and patients may derive a weight category for a subject by 1) taking raw measurements of height and weight, 2) deriving a Body Mass Index (“BMI”) for the subject based on the mathematical formula above, and then 3) correlating the BMI with a weight category using a table, such as Table 1 below. The measured height and weight would constitute raw anthropometric data while the BMI and weight category for the subject would constitute derived anthropometric data.

TABLE 1

| BMI in kg/m² | Weight Category |
|---|---|
| BMI < 18.5 | Underweight |
| 18.5 ≦ BMI < 25 | Normal weight |
| 25 ≦ BMI < 30 | Overweight |
| 30 ≦ BMI < 35 | Obesity class I |
| 35 ≦ BMI < 40 | Obesity class II |
| BMI ≧ 40 | Obesity class III (“morbid obesity”) |

In some instances, height and weight information pertaining to a subject could be entered into a software program that would then compute the BMI and associated weight category of the subject. Selected measurements chosen from the entered measurements are then presented to the user. Presentation of the measurements often takes the form of separate displays of the calculated BMI and/or weight category. These displays are effected by a simple textual presentation of the selected anthropometric data, and occasionally by separate scales for each selected metric that is sought to be displayed.

Although BMI has been prevalent in the medical community, laypeople have trouble understanding and getting used to this relatively new metric. Not only is it impractical to calculate the mathematical formula for BMI mentally, but subjects must also contend with the translation of their weight and height measurements to and from the metric system. Patients, as well as health care professionals, therefore must rely on tables, or calculators, to determine BMI.

The use of tables and calculators may simplify the mechanics of the calculation process, but they fail to provide a solution to the real issues facing patients. For example, a BMI of 25 kg/m² is just at the border between “normal” and “overweight,” according to the table above. A BMI of 30 kg/m² is at the border between “overweight” and “obese.” Patients in these situations are more interested in knowing the degree to which they exceed a weight norm or the amount of weight that they must lose in order to fall into a lower weight category. The BMI measure does not directly provide this information. In fact, while presently existing BMI calculators may provide the BMI and a weight category pertaining to a subject, there is no tool that outputs both the BMI and weight in an easy-to-understand, familiar, intuitive, and quantitative display.

Similarly, absolute maximal oxygen consumption (“VO2_{Max}”) is often used as a measure of a subject's physical fitness. Absolute maximal oxygen consumption represents the milliliters of oxygen that a person consumes when performing a standard physical activity, such as running on a treadmill, or using an exercise bike. Maximal oxygen consumption may be measured directly using an airflow measuring apparatus connected to the exercise machine or indirectly by measuring the subject's heart rate variability. Relative maximal oxygen consumption (“Rel. VO2_{Max}”) is obtained by dividing VO2_{Max} by a person's weight in kilograms. Medical literature uses Rel. VO2_{Max} in conjunction with a person's gender and age to arrive at a fitness category for the person, which ranges from “very poor” to “average” to “excellent”. A subject receiving this information, however, has no way of correlating his weight, which is controllable, with the fitness category measure. In other words, the subject may not be able to see how changes in weight will affect the subject's fitness category.

In general, this problem arises whenever a derived anthropometric measure such as BMI, or Rel. VO2_{Max} is used as a means to quantitatively express a particular characteristic, such as a weight category. This is because the subject only has control of the physical quantity (such as the weight) associated with the raw metric underlying the derived anthropometric measure (such as BMI, or Rel. VO2_{Max}). Therefore, subjects have an immediate interest in establishing a correlation between the controllable physical quantity that underlies the derived metric and the derived metric itself.

A display mechanism that allows subjects to intuitively and visually correlate a derived anthropometric measure with its underlying controllable raw anthropometric measure, would empower recipients of the information by giving them a practical means of tracking, monitoring and reaching health and wellness goals.

### SUMMARY

In accordance with the present invention, systems and methods for the display of anthropometric information are presented.

In some methods for the display of anthropometric information, raw anthropometric data pertaining to a subject is accepted and derived anthropometric measures based on the raw anthropometric data are calculated. A plurality of anthropometric data is then selected for display and the selected anthropometric data is displayed on a plurality of scales in an analog format using a single pointer. In some embodiments consistent with the present invention, the plurality of data selected for display may comprise of both raw and derived anthropometric data. In some embodiments consistent with the present invention, the raw data displayed may comprise the weight of a subject and the derived data displayed may comprise the BMI and the weight category of the subject. In some embodiments consistent with the present invention, the pointer may be aligned to a fixed scale corresponding to an anthropometric measure and other scales may be aligned with the pointer to correspond to the anthropometric measures represented by the individual scales. In some embodiments consistent with the present invention, some or all of the scales may be color-coded. These and other embodiments are further explained below with respect to the following figures.

### BRIEF DESCRIPTION OF THE DRAWINGS

FIG. 1 shows a block diagram of an anthropometric data visualization system consistent with some embodiments of the invention.

FIG. 2 illustrates a flowchart describing steps in a method for the display of a plurality of anthropometric data according to some embodiments of the invention.

FIG. 3A and FIG. 3B show a flowchart describing an exemplary system for the display of raw and derived anthropometric data of a subject according to some embodiments of the invention.

FIG. 4 shows a display screen for an exemplary circular scale, generated by a program that calculates and displays weight and BMI for a subject using a single pointer according to embodiments of the invention.

FIG. 5 shows an exemplary radial scale that displays weight, weight category, and BMI for a subject using a single pointer according to some embodiments of the invention.

### DETAILED DESCRIPTION

In some methods for anthropometric data visualization in accordance with the present invention, raw anthropometric data pertaining to a subject is accepted and/or measured. Derived anthropometric data is calculated based in part on the raw anthropometric data. In some embodiments consistent with the present invention, a plurality of the anthropometric data is then selected for display and the selected anthropometric data is displayed on a plurality of scales in an analog format using a single pointer.

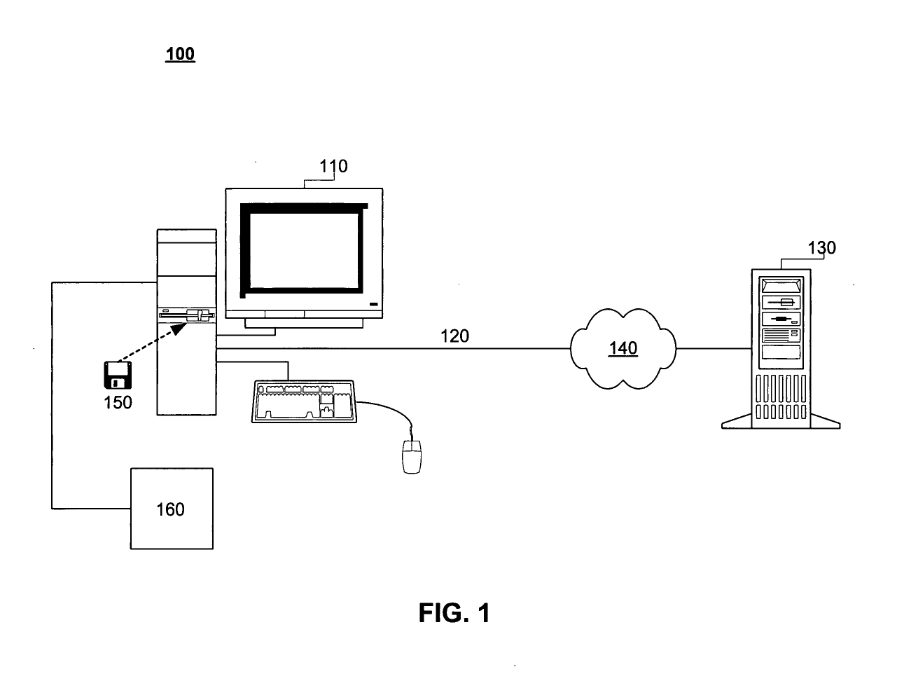

FIG. 1 illustrates an exemplary system 100 capable of displaying a plurality of anthropometric data according to the embodiments of the present invention. A computer software application consistent with the present invention may be deployed on a network of computers, as shown in FIG. 1, that are connected through communication links that allow information to be exchanged using conventional communication protocols and/or data port interfaces.

As shown in FIG. 1, exemplary system 100 includes a computing device 110 and a server 130. Further, computing device 110 and server 130 may communicate over a connection 120, which may pass through network 140, which in one case could be the Internet. Computing device 110 may be a computer workstation, desktop computer, laptop computer, personal data assistants, handheld computers, mobile phones with computing capabilities, or any other computing device capable of being used in a networked environment. Server 130 may be a platform capable of connecting to computing device 110 and other devices too (not shown).

Connection 120 couples computing device 110 and server 130, and may be implemented as a wired or wireless connection using conventional communication protocols and/or data port interfaces. In general, connection 120 can be any communication channel that allows transmission of data between computing device 110 and server 130, including network channels and transfer of data between machines on fixed storage media. In one embodiment, for example, both computing device 110 and server 130 may be provided with conventional data ports, such as ADB, USB, SCSI, FIREWIRE, AAUI, and/or BNC ports for transmission of data through the appropriate connection 120. In some embodiments, connection 120 may be a low-bandwidth connection, for example, a Digital Subscriber Line (DSL), an Asymmetric Digital Subscriber Line (ADSL), or a cable connection. The communication links could be wireless links or wired links or any combination consistent with embodiments of the present invention, that allows communication between computing device 110 and server 130.

Network 140 could include a Local Area Network (LAN), a Wide Area Network (WAN), the Internet, cellular phone networks, and/or cellular data networks. In some embodiments consistent with the present invention, information sent over network 140 may be encrypted to ensure the security of the data being transmitted.

Computing device 110 also contains removable media drive 150. Removable media drive 150 may include, for example, 3.5 inch floppy drives, CD-ROM drives, DVD ROM drives, CD±RW or DVD±RW drives, USB flash drives, and/or any other removable media drives consistent with embodiments of the present invention. In some embodiments consistent with the present invention, portions of the software application may reside on removable media and be executed by computing device 110 using removable media drive 150.

In some embodiments consistent with the present invention, System 100 may also contain a measuring device 160. Measuring device 160 may be used for the measurement of raw anthropometric data pertaining to a subject. Raw anthropometric data could include directly measurable physical quantities such as weight, or height, or maximal oxygen consumption. As shown in FIG. 1, measuring device 160 may be connected to computing device 110, through connection 120-2. In some embodiments consistent with the present invention, System 100 may contain multiple instances of measuring device 160. Each such measuring device could be used to make raw anthropometric measurements pertaining to different health or biological parameters for a subject. In some embodiments consistent with the present invention, raw anthropometric data could be directly entered into computing device 110 by an operator using an input device.

A computer software application consistent with the present invention may be deployed on any of the exemplary computers, as shown in FIG. 1. For example, computing device 110 could execute software that may be downloaded directly from server 130. In some embodiments consistent with the present invention, the software application for anthropometric data visualization may be distributed between the various computing systems shown in FIG. 1.

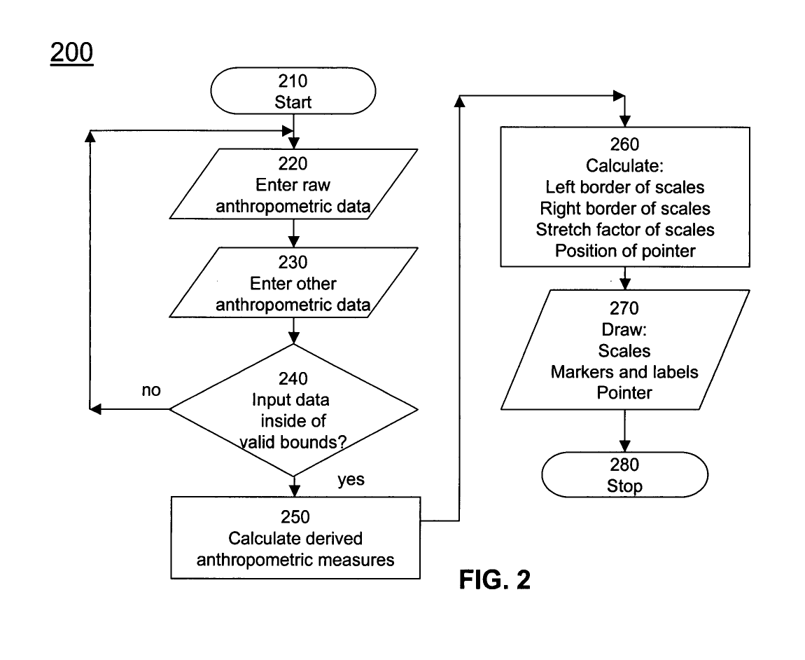

FIG. 2 illustrates a flowchart 200 describing steps in a method for the display of a plurality of anthropometric data according to some embodiments of the invention. A software application to perform steps in a method for the display of a plurality of anthropometric data is started in step 210, and raw anthropometric data is entered in step 220. Raw anthropometric data could include directly measurable physical quantities such as weight, or height, or maximal oxygen consumption. In some embodiments consistent with the present invention, portions of the software application may reside on computing device 110, server 130 or on removable media. In some embodiments consistent with the present invention, raw anthropometric data may be input directly from a measuring device and/or from other hardware or software components and/or manually entered by an operator. In some embodiments consistent with the present invention, data may be input from measuring devices such as exemplary measuring device 160. In step 230, other anthropometric data is input. Other anthropometric data could include data about sex, ethnicity, age, etc. that could be relevant in the calculation or categorization of derived anthropometric measures. For example, determining whether a certain anthropometric measure such as body fat percentage falls within a specific category such as normal, excessive etc. may depend on whether the subject is male or female. Other anthropometric data could be input by an operator and/or read from a file or from memory and/or input from another program. In step 240, input data is checked for consistency. While the algorithm is capable of displaying a plurality of scales without limitation, in practice, the range of values may be limited to correspond to realistic expectations. For example, it would be unrealistic to expect a person 2 feet tall weighing 800 pounds, or one 12 feet tall weighing 80 pounds. Accordingly, in some embodiments, the input ranges for anthropometric data may be reasonably limited based on expected ranges of input data. If input anthropometric data contains an error or is outside allowed ranges, the program returns to step 220, where the incorrect data may be re-entered. In step 250, if the data is correct, derived anthropometric measures are calculated. In step 260, display parameters are calculated for the plurality of data selected for display. In some embodiments consistent with the present invention, one of the scales may be fixed and the pointer set to point at the relevant value on the fixed scale. For each of the other scales, display parameters including limits for the scales and markings on the scales must be calculated based on the position of the pointer on that scale. For example, since the pointer position on a non-fixed scale represents the measured anthropometric value on that scale, the limits and markings on that scale must be calculated using the position of the pointer and the scale value at that position as a starting point. In some embodiments, mathematical relationships between the anthropometric measures may be exploited to quickly derive limits and markings for non-fixed scales. On a scale showing BMI, Weight and Weight category, for example, the ratio between BMI and Weight in kilograms (W) is constant for an adult subject and equal to the square of the adult subject's height (H) in meters. Therefore, if the BMI scale limits are used to derive the upper and lower limits of the weight scale then the distance between unit markings on the weight scale will need to be more tightly spaced than corresponding markings on the BMI scale by a factor of exactly (height)², in order for the pointer to line up at exactly the correct weight.

As an example:
if
18≦BMI≦40 on the BMI scale and H=2 meters, then
(18*H²)≦W≦(40*H²) or (18*4)kg≦W≦(40*4)kg.
Therefore,
18≦BMI≦40 corresponds to 72≦W≦160, on the W scale.

Thus, for the example above, if the W scale is drawn over the same length as the BMI scale with limits from 72 to 160 kilograms, and the distance separating 1 kilogram markings on the W scale is a quarter of the distance between 1 unit markings on the BMI scale, then the pointer will automatically point to the correct weight when drawn to point to the BMI measure. For example, if the BMI and W scales are drawn over the same length, and H=2 meters, then the distance from 18 to 19 on the BMI scale will need to be 4 times greater than the distance from 72 to 73 on the W scale, in order for the markings on the two scales to match and the pointer to be aligned correctly at the right measure on both scales. In some embodiments, a scale displaying weight, weight category and BMI using a single pointer may utilize the exemplary approach described above to calculate and display the measures.

In some embodiments consistent with the present invention, certain markings on the fixed scale may be positioned at a fixed visual angle. In some embodiments consistent with the present invention, the scales may be color-coded for easier readability. The plurality of scales with appropriate markings along with the single pointer are then displayed in step 260. For each scale a “stretch factor,” may be determined as the linear distance on the display corresponding to a unit increase in marker value on the scale. In some embodiments, the positions of markers on the display may be determined using the stretch factor. In some implementations, the user may be presented with several scale types, such as, for example, a linear scale type (with markings over straight lines), or a radial scale type (with markings over circular arcs) or a circular scale type (markings over the entire circumference of a circle), from which a particular scale type may be chosen for display. The chosen scale type is then used for each scale in the plurality of scales that are displayed in a manner consistent with embodiments of the present invention. In some embodiments, markers on a scale may serve to delimit categories. For example, a subject's weight category may be displayed on a scale based on the calculated BMI value. In this instance, markers on a weight category scale may simply be delimiters between weight categories such as “Underweight,” “Normal,” “Overweight,” or “Obese” that are displayed on the scale.

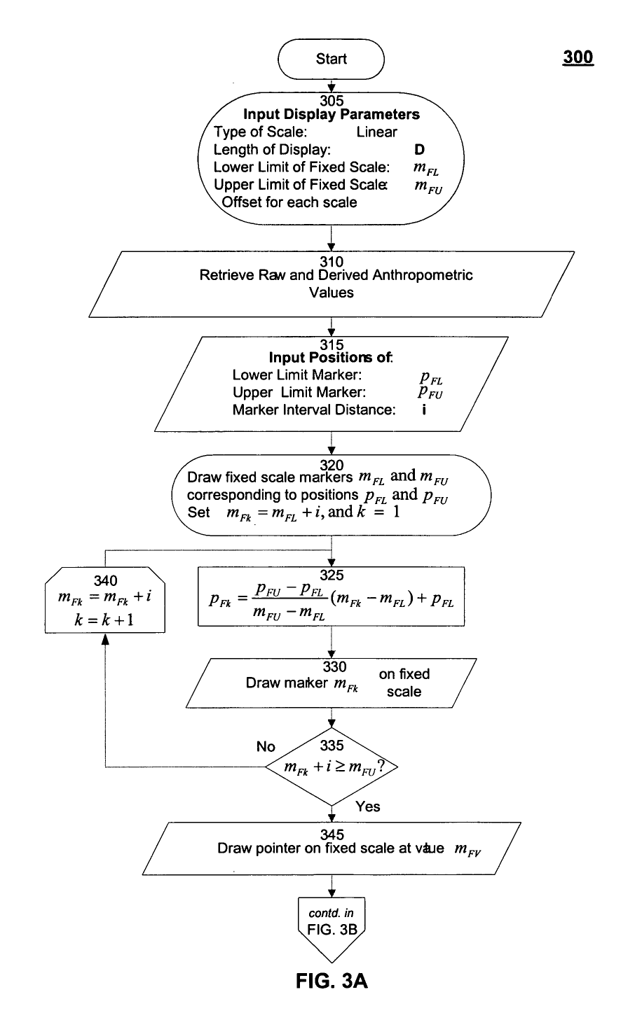

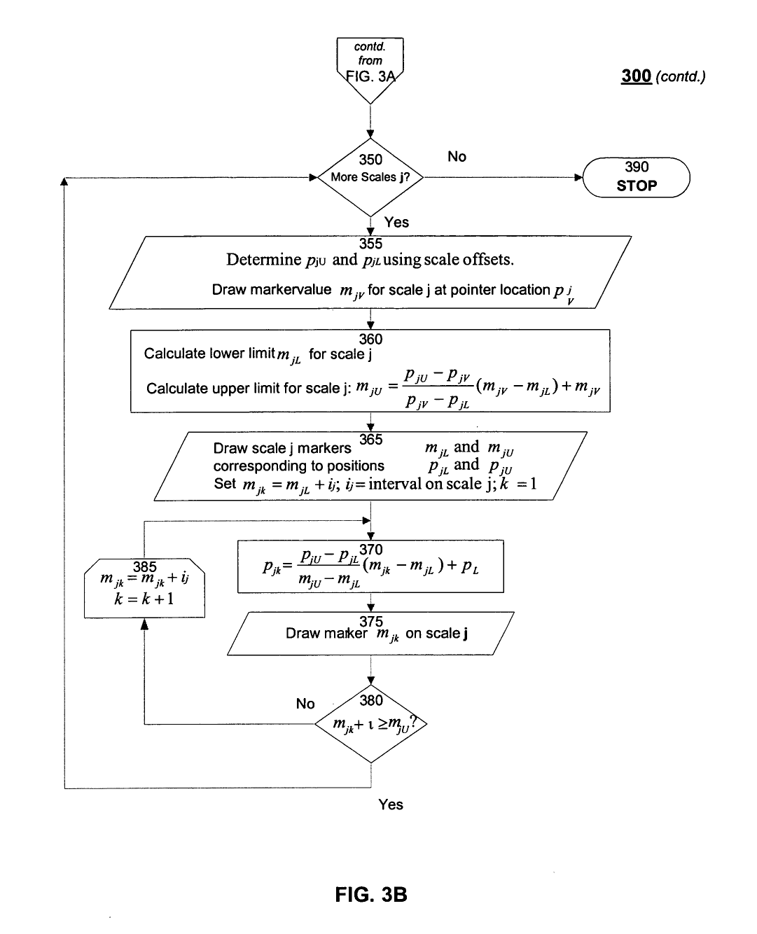

FIGS. 3A and 3B show a flowchart for an algorithm 300 for generating multiple scales with a single pointer according to embodiments of the present invention. A software application to perform steps in a method for the display of a plurality of anthropometric data is invoked and display parameters are entered in step 305. In some embodiments consistent with the present invention, display parameters may be input by users, read from a file, or passed to the application from another program. In some embodiments consistent with the present invention, portions of the software application may reside on computing device 110, server 130 or on removable media. In some embodiments, algorithm 300 may be implemented as a software module that is part of a software application implementing steps in flowchart 200.

In flowchart 300, the identifier “m” is used to refer to the values of the markers on a scale corresponding to anthropometric measurements, while the identifier “p” is used to refer to the positions of the markers on the display. The identifier “i” denotes the interval between markers on a scale. Subscripts to “m” and “p” identify the scale and marker in question. For example, p_{qr}, refers to the position of marker “q” on the scale “r.” The subscripts “L” and “U” denote the lower and upper limits of the identifiers. The subscript “V” refers to the value being displayed on a particular scale by the pointer.

Data input, in step 305, may include the type of scale, the length of the display D, the lower limit m_{FL}, and upper limit m_{FU} of the range of values displayed on the fixed scale, F, and the offset between the successive scales being displayed. In some embodiments, the offset, representing the distance between the scales, may be used to ensure that the scales do not overlap when displayed. In some embodiments, the type of scale may be linear, circular, radial or any other scale type according to embodiments of the invention. In some embodiments, a user may select a scale type from a menu of available scale types. In some embodiments, a scale type may be chosen by default. For the purposes of the exemplary algorithm described in flowchart 300, a linear scale is assumed.

In step 310, anthropometric values that have been input, measured, and/or calculated are retrieved. In some embodiments, the anthropometric values may be retrieved from memory, from storage, and/or received as parameters from another program. Anthropometric values are represented by markers on a scale, with a pointer indicating the displayed anthropometric value on each scale.

In step 315, the display positions of the lower limit marker, p_{FL}, and upper limit marker, P_{FU}, and the interval between markers, i, for the fixed scale are received. In some embodiments, the offset between successive scales may be used in conjunction with the positions of the upper and lower limit markers on a scale, to calculate the positions of corresponding upper and lower limit markers on succeeding scales.

In step 320, the upper and lower limit marker values, m_{FU} and m_{FL}, for the exemplary linear scale are drawn. Additionally, the next marker value, m_{Fk}, to be displayed on the scale is calculated as m_{Fk}=m_{Fl},+i, by adding the interval between markers to the value of the lower limit marker, m_{FL}. A counter k, that keeps track of markers, is initialized to 1.

In step 325, the position of the next marker value on the exemplary fixed linear scale is calculated. The position of the next marker value, p_{Fk}, is calculated as:

$${p}_{Fk} = \frac{{p}_{FU} - {p}_{FL}}{{m}_{FU} - {m}_{FL}} ( {m}_{Fk} - {m}_{FL} ) + {p}_{FL}$$

where, the ratio (p_{FU}−p_{FL})/(m_{FU}−m_{FL}) may be seen as a “stretch factor,” and determines the linear distance on the screen corresponding to a unit increase in marker value on the scale. In step 330, marker value m_{Fk}, calculated in step 320, is drawn at position p_{Fk}, calculated in step 325. In step 335, the next marker value, given by m_{Fk}+i, is compared with the upper limit of the marker value, m_{FU}. If m_{Fk}+i≦m_{FU}, then the current marker value is incremented, in step 340, the counter k is incremented, and the process returns to step 325. In some embodiments such as a scale divided into categories, the underlying metric used to determine the category may be used to generate markers at category transition points. For example, for a weight category scale, the underlying BMI marker value may be calculated and used to delimit a weight category at the appropriate position on the display. For example, at a position corresponding to a BMI marker value of 25, a marker may be drawn to delimit the “Normal” weight category from the “Overweight” weight category, according to Table 1.

If m_{Fk}+i>m_{FU} then, in step 345, a pointer is drawn pointing to the anthropometric value m_{Fv} for the fixed scale. The algorithm then proceeds to step 350, as shown in FIG. 3B.

If there are additional scales that need to be drawn then, in step 355, the display positions of the lower limit marker, p_{jL}, and upper limit marker, pⱼᵤ, of the next scale, j, are determined using scale offsets from step 305. Additionally, the marker value for scale j is drawn at pⱼᵥ, which is the position of the pointer on scale j.

Next, in step 360, the lower limit for marker value, m_{jL}, on scale j is calculated. In some embodiments, m_{jL} may be determined based on a formula used for the calculation of the derived anthropometric measures that are displayed using the scale. For example, if the height of a subject is 2 meters and the lower limit of a fixed BMI scale is 18 kilograms/meter², then the lower limit, W_{L}, on a weight scale may be calculated as W_{L}=(18*2²)=72 kilograms. In addition, for the exemplary linear scale type, in flowchart 300, the upper limit for marker value, m_{jU}, on scale j may be calculated as:

$${m}_{jU} = \frac{{p}_{jU} - {p}_{jV}}{{p}_{jV} - {p}_{jL}} ( {m}_{jV} - {m}_{jL} ) + {m}_{jV}$$

using the known values of p_{jU}, p_{jL}, p_{jL}, m_{jL}, and m_{jV}. In some embodiments, the upper limit for marker value, m_{jU}, may be determined first based on a formula used for the calculation of the anthropometric measures that are displayed using the scale. Subsequently, the lower limit may be determined by using known values of p_{jU}, p_{jL}, p_{jV}, m_{jU}, and m_{jV}.

In step 365, the upper limit marker value, m_{jU}, and lower limit marker value, m_{jL}are drawn at positions p_{jU} and p_{jL} respectively, on scale j. In addition, the value of the next marker mⱼₖ, on scale j, is calculated as mⱼₖ=m_{jL}+iⱼ, where iⱼ is the interval between markers for scale j.

In step 370, the position of the next marker value on scale j is calculated. The position of the next marker value, pⱼₖ, is calculated as:

$${p}_{jk} = \frac{{p}_{jU} - {p}_{jL}}{{m}_{jU} - {m}_{jL}} ( {m}_{jk} - {m}_{jL} ) + {p}_{jL}$$

where, (p_{jU}−p_{jL})/(m_{jU}−m_{jL}) is the stretch factor for scale j.

Next, in step 375, the marker mⱼₖ is drawn at position pⱼₖ scale j. In step 380, the next marker value, given by mⱼₖ+i, is compared with the upper limit of the marker value, m_{jU}, for scale j. If mⱼₖ+i≦m_{jU}, then the current marker value is incremented, in step 385. The counter k is also incremented, and the process returns to step 370.

In step 380, if mⱼₖ+i>m_{jU}then all of the markers for scale j have been drawn and the algorithm returns to step 350. If no additional scales remain to be processed, in step 350, the algorithm is terminated, in step 390.

In some embodiments, the scales, markers, pointers, and all other objects may be drawn in a memory buffer prior to being rendered on a display device.

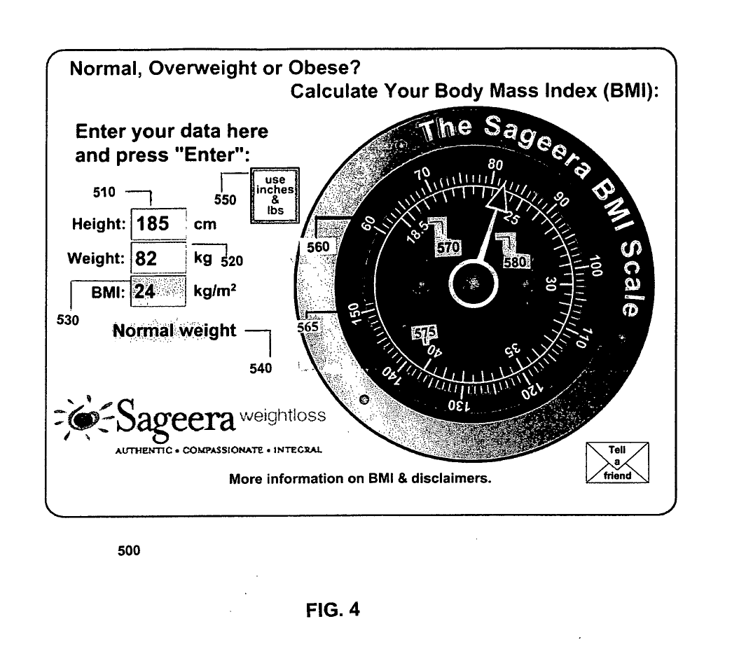

FIG. 4 shows a display screen for an exemplary circular scale 500, generated by a program that calculates and displays weight and BMI for a subject using a single pointer according to embodiments of the invention. In some embodiments, the raw anthropometric data pertaining to a subject may be entered at input locations 510 for height, and 520 for weight. Input boxes 510 and 520 can accept input in either metric units, or in feet/inches and pounds, or in British units such as stone (for weight). In general, input boxes 510 and 520 may be customized to accept data in a prevailing local standard for measurement. In some embodiments, a change from one set of units to another may be accomplished by selecting units on a unit selection box 550, which offers alternate choices for units. Once a user has entered valid data, the program calculates BMI and displays the BMI value on a circular scale in conjUnction with the weight value entered at location 520. The circular scale uses a single pointer 580, to display the entered and calculated values for weight and BMI. For example, in the embodiment shown in FIG. 4, pointer 580 indicates a value of 82 kilograms for the weight, and 24 kg/m² as the BMI value. The scales shown contain individual markers with selected values depicted. The exemplary BMI scale displayed has a range from lower limit 570 with a value 18.5 kg/m² to upper limit 575 with a value of 40 kg/m². The exemplary weight scale displayed has range from lower limit 560 with a value of 60 kg to upper limit 565 with a value of 150 kg. In some embodiments, BMI indicator 530 and weight category indicator 540 also display the BMI and weight category values independently.

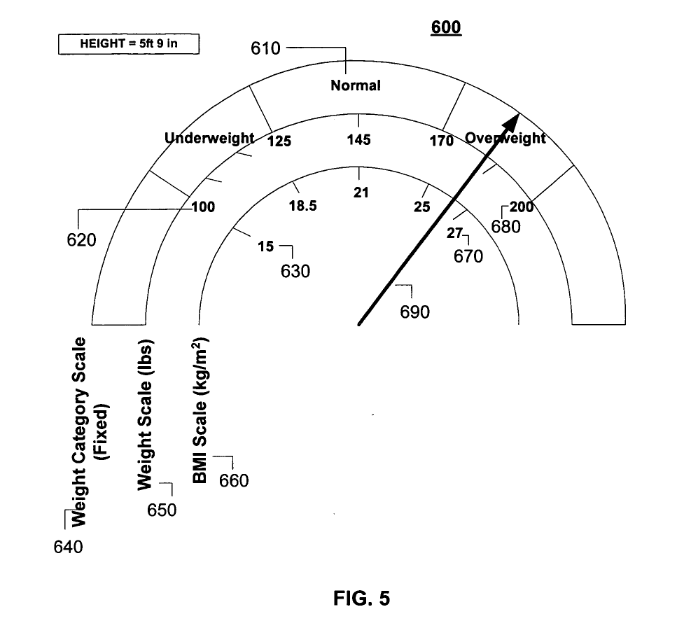

FIG. 5 shows an exemplary radial scale 600 that displays weight, weight category, and BMI for a subject using a single pointer according to embodiments of the invention. Exemplary scale 600 shows fixed weight category scale 640, weight scale 650 and BMI scale 660, with the measured, entered and/or calculated values for weight, weight category and. BMI indicated by single pointer 690. Weight category scale 640 has indicators using categories 610, such as “Normal,” “Underweight,”or “Overweight” to indicate weight category. Weight scale 650 and BMI scale 660 contain individual markers with selected values depicted. The exemplary weight scale displayed has a range from lower limit 620 with a value of 100 lbs. to upper limit 680 with a value of 200 lbs. The exemplary BMI scale displayed has a range from lower limit 630 with a value 15 kg/m² to upper limit 670 with a value of 27 kg/m². In exemplary scale 600, as shown in FIG. 6, a weight category indicator may appear at a certain visual angle. For example, the “Overweight” category indicator appears to the right of the display. Exemplary scale 600 allows a user to intuitively and visually correlate raw anthropometric measures, such as weight, with derived anthropometric measures such as weight category, or BMI.

Further, methods consistent with embodiments of the invention may conveniently be implemented using program modules, hardware modules, or a combination of program and hardware modules. Such modules, when executed, may perform the steps and features disclosed herein, including those disclosed with reference to the exemplary flow charts shown in the figures. Embodiments of the invention may also relate to different types of anthropometric measures and/or scale types. The methods and algorithms described in the specification and examples may be extended and/or modified to additional measures and/or scale types. The operations, stages, and procedures described above and illustrated in the accompanying drawings are sufficiently disclosed to permit one of ordinary skill in the art to practice the invention. Moreover, there are many computers and operating systems that may be used in practicing embodiments of the instant invention and, therefore, no detailed computer program could be provided that would be applicable to these many different systems. Each user of a particular computer will be aware of the language, hardware, and tools that which are most useful for that user's needs and purposes.

The above-noted features and aspects of the present invention may be implemented in various environments. Such environments and related applications may be specially constructed for performing the various processes and operations of the invention, or they may include a general-purpose computer or computing platform selectively activated or reconfigured by program code to provide the finctionality.

Embodiments of the present invention also relate to computer-readable media that include program instructions or program code for performing various computer-implemented operations based on the methods and processes of embodiments of the invention. The program instructions may be those specially designed and constructed for the purposes of the invention, or they may be of the kind well known and available to those having skill in the computer software arts. Examples of program instructions include, for example, machine code, such as produced by a compiler, and files containing a high-level code that can be executed by the computer using an interpreter.

Other embodiments of the invention will be apparent to those skilled in the art from consideration of the specification and practice of the embodiments of the invention disclosed herein. It is intended that the specification and examples be considered as exemplary only, with a true scope and spirit of the invention being indicated by the following claims. As such, the invention is limited only by the following claims.

1. A method of simultaneously displaying multiple anthropometric measures on a device, the method comprising:
accepting anthropometric data pertaining to a subject;
calculating derived anthropometric measures based on the accepted data;
selecting a plurality of anthropometric data for display; and
generating a scale corresponding to each selected anthropometric data; and
indicating the values of the selected anthropometric data on their respective scales in an analog format, using a single pointer.

2. The method of claim 1 wherein accepting anthropometric data pertaining to a subject comprises:
accepting raw anthropometric data pertaining to a subject; and/or
accepting other anthropometric data of the subject.

3. The method of claim 1, wherein selecting a plurality of anthropometric data for display comprises choosing at least one each of the raw anthropometric data, and the derived anthropometric measures.

4. The method of claim 1, wherein indicating the values of the selected anthropometric data on their respective scales in an analog format, using a single pointer further comprises aligning a marker on each scale corresponding to the value of anthropometric data for that scale, with a display window.

5. The method of claim 1, wherein indicating the values of the selected anthropometric data on their respective scales in an analog format, using a single pointer further comprises aligning a marker on each scale corresponding to the value of anthropometric data for that scale, with the single pointer.

6. The method of claim 1, wherein indicating the values of the selected anthropometric data on their respective scales further comprises generating images of the individual scales and the pointer.

7. The method of claim 1, wherein indicating the values of the selected anthropometric data on their respective scales in an analog format, using a single pointer further comprises:
aligning the single pointer to a value on a fixed scale corresponding to an anthropometric data selected for display; and
aligning the values on other scales corresponding to the remaining anthropometric data selected for display with the single pointer.

8. The method of claim 7 wherein the fixed scale may be color-coded.

9. The method of claim 1, wherein:
the accepted anthropometric data includes the height of the subject;
the accepted anthropometric data includes the weight of the subject; and
the derived anthropometric measures include the body mass index.

10. The method of claim 9, wherein the derived anthropometric measures further include the weight category of the subject.

11. The method of claim 10, where the weight category is selected from one of several weight categories based on the deviation of the weight of the subject from a norm.

12. The method of claim 10, where the weight category is chosen from one of underweight, normal, overweight, or obese.

13. The method of claim 1, wherein:
the accepted anthropometric data includes the age of the subject;
the accepted anthropometric data includes the gender of the subject;
the accepted anthropometric data includes the weight of the subject;
the accepted anthropometric data includes the maximum oxygen consumption of the subject; and
the derived anthropometric measures include the relative maximum oxygen consumption of the subject.

14. The method of claim 13, wherein the derived anthropometric measures further include the cardio-respiratory fitness category of the subject.

15. The method of claim 14, where the fitness category is selected from one of several cardio-respiratory fitness categories based on the deviation of the relative maximum oxygen consumption of the subject from a norm.

16. The method of claim 15, where the cardio-respiratory fitness category is chosen from one of very poor, poor, fair, average, good, very good and excellent.

17. An apparatus for simultaneously displaying multiple anthropometric measures on a device comprising:
means for accepting anthropometric data pertaining to a subject;
means for calculating derived anthropometric measures based on the accepted anthropometric data;
means for selecting anthropometric data for display;
means for generating a scale for each selected anthropometric data; and
means for indicating the anthropometric data value on each of the scales in an analog format using a single pointer.

18. The apparatus of claim 17, wherein means for accepting anthropometric data further comprises:
means for accepting raw anthropometric data; and/or
means for accepting other anthropometric data.

19. The apparatus of claim 17, wherein means for indicating the anthropometric data value on each of the scales in an analog format using a single pointer further comprises means for aligning a marker on each scale corresponding to the value of anthropometric data for that scale, with a display window.

20. The apparatus of claim 17, wherein means for indicating the anthropometric data value on each of the scales in an analog format using a single pointer further comprises means for aligning a marker on each scale corresponding to the value of anthropometric data for that scale, with a pointer.

21. The apparatus of claim 17, wherein means for indicating the anthropometric data value on each of the scales in an analog format further comprises means for generating images of the individual scales and a pointer.

22. The apparatus of claim 17, wherein means for indicating the anthropometric data value on each of the scales in an analog format using a single pointer further comprises:
means for aligning a single pointer to a value on a fixed scale corresponding to one of the anthropometric data selected for display; and
means for aligning the values on other scales corresponding to the remaining anthropometric data selected for display with the pointer.

23. A method of simultaneously displaying multiple anthropometric measures on a device, the method comprising:
accepting height data of a subject;
measuring the weight of the subject;
calculating derived anthropometric measures based on the height and weight data of the subject;
selecting weight data, and at least one of the derived measured anthropometric measures for display;
generating a scale for weight, and a scale for each of the selected derived anthropometric measures; and
indicating the value of the weight, and the values of each of the selected derived anthropometric measures of the subject on their respective scales in an analog format, using a single pointer.

24. The method of claim 23, wherein calculating derived anthropometric measures comprises computation of the body mass index of the subject.

25. The method of claim 23, wherein calculating derived anthropometric measures comprises computation of the weight category of the subject.

26. A method of simultaneously displaying multiple anthropometric measures on a device, the method comprising:
accepting the age of a subject;
accepting the gender of the subject;
measuring the weight of the subject;
measuring the maximum oxygen consumption of the subject;
calculating derived anthropometric measures based on some or all of the accepted and measured data of the subject; and
selecting the weight, the maximum oxygen consumption, and at least one of the derived anthropometric measures of the subject for display;
generating a scale for weight, a scale for the maximum oxygen consumption, and a scale for each of the selected derived anthropometric data; and
indicating the values for weight of the subject, the maximum oxygen consumption of the subject, and values for each of the selected derived anthropometric measures of the subject on their respective scales in an analog format, using a single pointer.

27. The method of claim 26, wherein calculating derived anthropometric measures comprises computation of the relative maximum oxygen consumption of the subject.

28. The method of claim 26, wherein calculating derived anthropometric measures comprises computation of the fitness category of the subject.

29. A method of simultaneously displaying multiple anthropometric measures on a device, the method comprising:
accepting raw anthropometric data pertaining to a subject;
calculating derived anthropometric measures based on the raw anthropometric data;
selecting a plurality of anthropometric data for display;
generating a scale for each selected anthropometric data; and
indicating the values of the selected anthropometric data on their respective scales in an analog format, using a single pointer.

30. The method of claim 29, wherein selecting anthropometric data for display comprises choosing at least one each of the raw anthropometric data and the derived anthropometric measures.

31. The method of claim 29, wherein indicating the values of the selected anthropometric data on their respective scales in an analog format, using a single pointer further comprises aligning a marker on each scale corresponding to the value of anthropometric data for that scale, with a display window.

32. The method of claim 29, wherein indicating the values of the selected anthropometric data on their respective scales in an analog format using a single pointer further comprises aligning a marker on each scale corresponding to the value of anthropometric data for that scale, with the single pointer.

33. The method of claim 29, wherein indicating the values of the selected anthropometric data on their respective scales in an analog format using a single pointer further comprises generating images of the individual scales and the single pointer.

34. The method of claim 29, wherein indicating the values of the selected anthropometric data on their respective scales in an analog format using a single pointer further comprises:
aligning the single pointer to a value on a fixed scale corresponding to an anthropometric data selected for display; and
aligning the values on other scales corresponding to the remaining anthropometric data selected for display with the pointer.

35. The method of claim 34 wherein the fixed scale may be color-coded.

36. The method of claim 29, wherein:
the accepted raw anthropometric data includes the height of the subject;
the accepted anthropometric data further includes the weight of the subject; and
the derived anthropometric data includes the body mass index.

37. The method of claim 36, wherein the derived anthropometric data further includes the weight category of the subject.

38. The method of claim 37, where the weight category is selected from one of several weight categories based on the deviation of the weight of the subject from a norm.

39. The method of claim 29, wherein:
the accepted raw anthropometric data includes the age of the subject;
the accepted raw anthropometric data further includes the gender of the subject;
the accepted anthropometric data further includes the weight of the subject;
the accepted anthropometric data further includes the maximum oxygen; consumption of the subject; and
the derived anthropometric data includes the relative maximum oxygen consumption of the subject.

40. The method of claim 39, wherein the derived anthropometric data further includes the cardio-respiratory fitness category of the subject.

41. The method of claim 40, where the fitness category is selected from one of several cardio-respiratory fitness categories based on the deviation of the relative maximum oxygen consumption of the subject of the subject from a norm.

42. A computer-readable medium that stores instructions, which when executed by a computer perform steps in a method for displaying anthropometric data, the steps comprising:
accepting raw anthropometric data pertaining to a subject;
calculating derived anthropometric measures based on the raw anthropometric data;
selecting a plurality of anthropometric data for display;
generating a scale for each selected anthropometric data; and
indicating the values of the selected anthropometric data on their respective scales in an analog format, using a single pointer.

43. The method of claim 42, wherein accepting raw anthropometric data pertaining to a subject comprises accepting anthropometric data input from a measuring device.

44. The method of claim 42, wherein accepting raw anthropometric data pertaining to a subject comprises accepting anthropometric data input from a user.

45. The method of claim 42, wherein accepting raw anthropometric data pertaining to a subject comprises reading anthropometric data stored in machine-readable form.

46. The method of claim 42, wherein selecting a plurality of anthropometric data for display is based on predefined criteria stored by a user.

47. The method of claim 42, wherein displaying the selected anthropometric data on a plurality of scales in an analog format using a single pointer is performed on a monitor.

48. A computer readable memory containing instructions for controlling a computer system to perform steps in a method for displaying anthropometric data, the steps comprising:
accepting raw anthropometric data pertaining to a subject;
calculating derived anthropometric measures based on the raw anthropometric data;
selecting a plurality of anthropometric data for display;
generating a scale for each selected anthropometric data; and
indicating the values of the selected anthropometric data on their respective scales in an analog format, using a single pointer.

49. A computer processor executing instructions that performs steps in a method for displaying anthropometric data, the steps comprising:
accepting raw anthropometric data pertaining to a subject;
calculating derived anthropometric measures based on the raw anthropometric data;
selecting a plurality of anthropometric data for display;
generating a scale for each selected anthropometric data; and
indicating tie values of the selected anthropometric data on their respective scales in an analog format, using a single pointer.
